(19) United States
(12) Patent Application Publication
(10) Pub. No.: US 2006/0240456 A1
(43) Pub. Date: Oct. 26, 2006

(54) Nanosized biological container and manufacture thereof

(76) Inventors: Liaohai Chen, Darien, IL (US); Samuel D. Bader, Oak Park, IL (US); Axel F. Hoffmann, Chicago, IL (US); Brian K. Kay, Chicago, IL (US); Lee Makowski, Hinsdale, IL (US)

Correspondence Address: BARNES & THORNBURG LLP, P.O. BOX 2786, CHICAGO, IL, 60690-2786, US

(21) Appl. No.: 11/384,792

(22) Filed: Mar. 20, 2006

Related U.S. Application Data
(60) Provisional application No. 60/664,235, filed on Mar. 22, 2005.

(51) Int. Cl. C12Q 1/70; C12Q 1/68

(52) U.S. Cl. 435/6; 435/5

(57) ABSTRACT

Nanosized biological containers that are “ghosts” of viruses for which capsids are independent of their endogenous viral nucleic acid cores, provide nano-particles of uniform size, and known numbers of sites for attachments of ligands. These containers can be filled with a fluorescent, magnetic, x-ray absorbent, nucleotide components or a radioactive particle and used as nanoscale markers.

### RELATED APPLICATIONS

This application claims priority under 35 USC §119(e) to U.S. Provisional Application Ser. No. 60/664,235, filed Mar. 22, 2005, the disclosure of which is incorporated herein by reference.

### U.S. GOVERNMENT RIGHTS

The United States Government has rights in this invention pursuant to Contract W-31-109-ENG-38 between the U.S. Department of Energy (DOE) and the University of Chicago representing Argonne National Laboratory.

### BACKGROUND

Nanosized biological containers that are “ghosts” of viruses for which capsids are independent of their endogenous viral nucleic acid cores, provide nano-particles of uniform size, and known numbers of sites for attachments of ligands.

Biological architectures have attracted attention as spatially-defined templates for fabricating mono-dispersed nano-particles. For example, the apo form of the ferritin protein has been used as a template (protein cage) to fabricate magnetic particles at the nanometer scale; and bacteria have been used as a host to accommodate the formation of polymer material on the micrometer scale for drug delivery. Lipid assemblies, DNA, and multicellular superstructures have been used to direct the patterning and deposition of inorganic material in the micro-to-nanometer scale, compatible with biological entities ranging from proteins to cells and bacteria.

Viruses exemplify an extraordinarily organized nano-architecture which can be harnessed as templates for material synthesis. Viruses are complex molecular biosystems in which nucleic acid strands are confined within a nano-sized (10˜500 nm) compartment (capsid). The function of the capsid is to provide a rigid and robust container protecting the nucleic acids in the passage from one host cell to another and to deliver the nucleic acid to the appropriate site. Thus, capsid proteins often have the capacity to be self-assembled into hollow cages in vitro. Indeed, in vitro self-assembled protein cages derived from plant viruses have been used as containers to host inorganic mineralization.

### SUMMARY

A nanosized biological container is formed by removing endogenous DNA or RNA from a natural viral capsid protein shell and placing an exogenous functional core inside the shell. The exogenous functional core can be selected from the group consisting of a fluorescent, magnetic, x-ray absorbent, non-native nucleotide entities, and radioactive particles. For biological applications, containers should be non-toxic, stable in aqueous solutions at neutral pH, and resistant to biodegradation. Suitably tailored virus have capsids independent of nucleic acid cores.

A suitable nanosized biological container is made from the viral capsid protein shell of a T7 bacteriophage, that is, a ghost.

The exogenous functional core is selected from the group consisting of a fluorescent, magnetic, x-ray absorbent, nucleotide and radioactive particle.

A suitable magnetic particle is cobalt.

Using magnetic field gradients, the magnetic particles can be subjected to significant forces even when they are embedded in a biological environment. Accordingly a biosensor can be provided wherein a nanosized biological container has a magnetic exogenous functional core, and said nanosized biological container is covalently bound to a solid support via a linker. Magnetic particles in T7 phage are spherical and about 40 nm in diameter.

A suitable fluorescent particle is a lonthonide complex.

A biosensor is formed from the nanosized biological container described herein, and an exogenous functional core suitable for biosensing a target, such as B. subtilis spores.

The sensing mechanism relies on a modification of the dynamic properties of the magnetic virus, after it is bound to its target molecule. Namely, by binding the virus with a flexible linker molecule to a substrate, a simple oscillating system with a distinct resonance frequency, can be probed by ac-susceptibility measurements. This approach has the advantage that there is no need for tagging the target molecule, since the binding sites on the virus have already been selected for high affinity and specificity. Furthermore the sensing mechanism has an internal check for integrity, since a malfunction will be recognized easily by an absence of any resonance signal.

The capsid protein shell of the nanosized biological containers can be modified to include ligands that are covalently bound to the external surface of the capsid shell. More particularly, the capsid protein shell may represent a fusion protein, wherein the fusion protein includes the ligand. The ligand may be an antibody or other protein capable of specific binding to another molecule. The capsid protein shell may also be provided with a linker that allows the nanosized biological container to be bound to a solid support.

A plurality of nanosized containers is within the scope of the disclosure.

A method of preparing a nanosized biological container includes the steps of:

(a) purifying viral cells (phage with capsids independent of the endogenous nucleic acid cores) by centrifugation using cesium chloride density gradients, and

(b) treating the purified cells with alkaline buffer.

A method of preparing a nanosized biological container includes the steps of:

(a) contacting phage with a sodium sulfate solution in the presence of DNAase; and

(b) purifying phage ghosts by centrifugation using cesium chloride density gradients.

A method of preparing a nanosized biological container, the method comprising:

(a) contacting phage with with alkaline buffer;

(b) isolating the capsid proteins;

(c) renaturing the capsid proteins to form phage ghosts.

A method of placing an exogenous core into a viral capsid includes the steps of:

(a) obtaining a solution of capsid without endogenous cores; and

(b) mixing a solution of endogenous core particles with the solution of empty capsids.

A method of adding ligands to the surface of a viral shell includes the steps of

(a) obtaining a solution of viral shells (phage ghosts); and

(b) adding ligands such that the ligands are bound to the viral shell viral capsids.

A method of manufacturing uniform nanosized particles with a uniform size distribution, includes the steps of:

(a) obtaining a solution of phage ghosts displaying ligands; and

(b) selecting the phage ghosts with ligands.

A method of performing an enzyme linked immunosorbant assay (ELISA) using nanosized containers is provided, the method including the steps of:

(a) preparing microtiter plates containing protein;

(b) adding nanosized containers of the present disclosure to the wells;

(c) adding labeled protein to the wells; and

(d) interpreting the results to determine binding.

A hybridized phage probe was constructed using certain tailed phages that build their protein shells first and subsequently condense the nucleic acid within them. As a result, an empty capsid (“ghost phage” or “pseudo phage”) from the tailed phage is stable without interior endogenous DNA. The architecture of a ghost phage can function as a unique nano-container for uniform fabrication of a nano-sized functional particle (e.g. fluorescent, magnetic, radioactive, or the like) inside. Consequently, such a hybridized system contains a functional core and a capsid protein shell.

Novel magnetic viruses, which exemplify a new approach to the synthesis of hybrid inorganic/biological materials were constructed. In an embodiment, tailed icosahedral T7 bacteriophage was used to fabricate a hybridized phage. Structurally T7 phage consists of a capsid shell, a head-tail connector, tail, and tail fibers. The morphogenesis of T7 and its DNA package have been well documented by Studier (1969, 1972). Empty capsid shells of T7 were assembled prior to the DNA packaging and were isolated at the early stage of the lytic infection. Given the diameter of T7 phage (approximately 55 nm); a ghost T7 phage provided a cavity of ˜40 nm, which was used to accommodate fluorescent materials, thus, leading to a fluorescent material hybridized T7 phage.

Novel magnetic viruses, which exemplify a new approach to the synthesis of hybrid inorganic/biological materials were constructed. For the synthesis, empty T7 phage were generated by alkaline treatment, which was subsequently used as a template to fabricate cobalt metal nano-particles. The resulting magnetic viruses had uniformly sized cobalt particles of 42±2 nm diameter. Furthermore, the bio-functionality is also uniform with 415 copies of ligand attached to each magnetic virus, which can be manipulated by using different types of phage vectors for the starting material.

The use of phage capsids as the template for fabrication of materials provides a dramatically new way to functionalize nano-particles with affinity reagents or to tag an affinity reagent with magnetic nano-particles. This new system can serve as a biosensor. Another value is that it is a unique method to approach a major challenge in nanoscience-precision and reproducibility. Uniform particle size distributions result, and the containers have specific sites for ligand attachments. It is contemplated that the magnetic and bio-functional phage can be assembled precisely using a shaped bio-architecture and bio-recognition force. In addition to biomedical applications, such as biosensing, target reagent delivery, magnetic hypothermal treatments, and MRI contrast enhancement, virus-templated nanoparticles will benefit the creation of nanomaterials in general.

Libraries of T7 virions displaying cDNA expression products or single-chain antibodies are screened to select members which bind to B. subtilis spores (coat protein CotE), which serve as a model for B. anthracis spores.

2D “magnetic-virus” arrays are assembled using self-assembled monolayer approaches.

Drugs can be released from the nanosized container. In addition it is anticipated that the present nanoparticles can be usedDevelop a strategy for single-molecule detection using either a force detection or integrated induction coil approach.

### DEFINITIONS

A “linker” as used herein is a molecule, or group of molecules, attached to a substrate that spaces a biologic material from the substrate. Linkers may further supply a labile linkage that allows a biologic material to be detached from the substrate. Labile linkages include photocleavable groups, acid-labile moieties, base-labile moieties and enzyme-cleavable groups.

As used herein the term “solid support” relates to a solvent insoluble substrate that is capable of forming linkages (e.g. covalent bonds) with molecules. The support can be either biological in nature, including but not limited to, a cell or bacteriophage particle, or synthetic, including but not limited to, an acrylamide derivative, glass, plastic, agarose, cellulose, nylon, silica, or magnetized particles. The surface of such supports may be solid or porous and of any convenient shape.

As used herein a ligand is any molecule that binds to another molecule, and more particularly a protein ligand is an atom, a molecule (i.e. another protein) or an ion which can bind to a specific site on a protein.

As used herein, the term “antibody” refers to a polypeptide or group of polypeptides which are comprised of at least one binding domain. Antibodies include recombinant proteins comprising a binding domain (including single-chain antibodies), as well as fragments, including Fab, Fab′, F(ab)2, and F(ab′)2 fragments.

As used herein the term “functional core” refers to a particle, compound or other moiety that has dimensions suitable for being packaged within a viral capsid shell while retaining its desired functional characteristics.

An “exogenous functional core” defines the relationship between a particular viral capsid shell and a functional core, wherein an “exogenous functional core” is one that is not naturally associated with the particular capsid shell.

### BRIEF DESCRIPTION OF THE DRAWINGS

FIG. 1A shows the fluorescence spectrum of europium complex hybridized T7 phage; FIG. 1B shows results of a time-resolved fluorescence immunosorbent assay.

FIG. 2 shows an energy dispersive X-ray (EDX) spectrum indicating the dominated europium element inside T7 ghosts.

FIG. 3 shows an energy dispersive X-ray (EDX) analysis, indicating the dominant element inside the T7 ghost was determined to be cobalt.

FIG. 4A and FIG. 4B shows a schematic view demonstrating the interaction of the various components during an enzyme linked assay; FIG. 4B shows the : ELISA result for the cobalt hybrid T7 phage.

FIG. 5 shows the confirmation of binding activity of ghost phage using S-protein ELISA.

FIG. 6 shows a schematic illustration for 2D assembly of “magnetic virus”.

FIG. 7 shows a schematic of sensing principles.

FIG. 8 illustrates phage-displayed to a protein target. Affinity selection of phage from a library involves incubating phage in microtiter plate wells (A) which have been coated with target protein, washing away unbound phage (B), releasing the bound phage with denaturation of the target protein (C), infecting bacterial with phage recovered from the wells (D), and incubating the amplified particles with target once again (E). This overall process is typically repeated two to three additional times, before plating the viral particles at a limiting dilution to give individual plaques on a bacterial lawn in a petri plate (F).

### DETAILED DESCRIPTION

Synthesis of controllable nano-scale magnetics and their integration with biological systems is a goal. Magnetic nano-materials are synthesized within an empty ligand-displayed phage virus. The empty virus architecture provides an excellent, spatially-defined host system which can be harnessed as a template for uniform fabrication of magnetic nanoparticles. In addition, the use of ligand-displayed phage capsid as the template for particle synthesis has the advantage that the size and shape of the particle can be highly regulated through bio-engineering of the capsid. Benefiting from the phage display technology, the particle generated inside the phage has integrated bio-recognition elements with high affinity and specificity for selected target molecules. This approach challenges the traditional view that materials' synthesis is confined to the thermodynamics of “condensed matter”. Instead, it offers a new approach of “synthesis with design” and revolutionary way to “tag” or “functionalize” the magnetic nano-particles.

A nanosized biological container is provided that has been functionalized to display a ligand on its external surface that interacts with a target, wherein the container further includes an exogenous functional core that serves as a detectable marker. The interaction between the target and the ligand can include a binding interaction. The nanosized container can be covalently bound to a solid support, and can be bound through a linker.

Nanosized biological containers that are “ghosts” of viruses for which capsids are independent of their endogenous viral nucleic acid cores, provide nano-particles of uniform size, and known numbers of sites for attachments of ligands. T7 bacteriophage particles are especially well suited to be used as templates for inorganic nano-particles. Although many viruses build their capsids and encapsulate their nucleic acids either by co-condensation or by condensing the nucleic acid first and subsequently building a protein shell around it, some viruses, such as tailed T7 phage (a fast growing and extremely stable double-strand DNA phage), build their protein shells first and subsequently condense the nucleic acids within them. As a result, an empty T7 capsid (“ghost virus”) is stable without interior DNA. This is confirmed by the fact that ghost T7 particles are always present in bacterial cultures infected with T7 phage. The outer diameter of the T7 phage is of the order of 55 nm; thus, a T7 ghost can provide a cavity of ˜40 nm in the absence of its DNA, which can serve as a template for fabrication of nano-sized particles.

Fluorescent probes play important roles in biology, medicine, and biotechnology. Conventionally, a fluorescent probe is made by chemically conjugating a fluorophore molecule or a quantum dot to a biomolecule (such as an affinity reagent). A few unconventional probes have been developed. Researchers have explored the use of Aequorea-derived fluorescent proteins (AFP). As an alternative to AFP, tetracysteine-biarsenical labeling also has been reported as a very useful probing tool. In both cases, the “fluorophores” (fluorescent proteins, such as AFP and tetracysteine-biarsenical complex) are fused to the affinity regents by molecular biology methods instead of chemical conjugation.

A novel fluorescent probe of europium-complex hybridized T7 phage was made by filling a ligand displayed T7 ghost phage with fluorescent europium complex. The structure of the hybridized phage, which contains a fluorescent inorganic core surrounded by a ligand displayed capsid shell, was confirmed by electron microscope, energy dispersive X-ray analysis (EDX), bioassays, and fluorescence spectrometer. As a benefit of the phage display technology, the hybridized phage has the capability to integrate an affinity reagent against virtually any target molecule. This approach provides an original method to fluorescently “tag” a bio-ligand and to “bio-fimctionalize” a fluorophore particle. By using other types of materials such as radioactive or magnetic materials to fill the ghost phage, the hybridized phages represent a new class of fluorescent, magnetic, or radio probes, useful both in vitro and in vivo applications.

An advantage of using fluorescent material hybridized T7 phage as a fluorescence probe is that this composition has the capability to carry a specific affinity reagent against virtually any target molecules via phage display technology. In phage display, ligands (such as recombinant antibody fragments, cDNA-encoded segments, or combinatorial peptide chains) are expressed as fusions to a capsid protein present on the surface of viral particles.

The biological functionality of T7 phage can be tailored by generating affinity reagents through phage display technology. In phage-display, molecules such as antibody fragments, cDNA encoded segments, or combinatorial peptides are expressed as fusions to a capsid protein present on the surface of viral particles. Libraries of millions to billions of phage particles, each displaying a different fusion protein, are screened (usually by affinity selection) for members displaying the desired properties or binding affinities. Phage display offers the following advantages: (a) the peptide or proteins which are expressed on the surface of the viral particles are accessible for interactions with their targets; (b) the recombinant viral particles are highly stable; (c) the viruses can be amplified (grown), and (d) each viral particle contains the DNA encoding its recombinant genome, thereby providing a physical linkage between the genotype and phenotype. Thus, phage libraries can be methodically screened by isolating viral particles that bind to targets, plaque-purifying the recovered phage, and sequencing the phage DNA inserts. Usually three rounds of affinity selection are sufficient to isolate tightly binding phage. Virtually any affinity reagent specific to any target can be displayed on a T7 phage surface via phage display. Both wild type as well as S-tag peptide, a short peptide with nanomolar affinity to a fragment of RNAse A, display T7 phage.

Among the fluorophores used in the fluorescent probes, the lanthanide complex (such as europium or terebium complex) exhibits unique spectral characteristics. Lanthanide complexes are superior to conventional organic fluorophores because they have very sharp emission spectra with more than a 300-nm Stoke shift, plus an extremely long fluorescence lifetime (˜millisecond), which can be easily detected by time-resolved fluorescence spectroscopy. Thus, lanthanide complexes are the ideal fluorophores to overcome the problems associated with background fluorescence in a biological sample. However, making a lanthanide probe, like making other fluorescence probes, involves chemically conjugating a ligand molecule (affinity reagent) to the chelator molecule, which requires cumbersome synthetic efforts.

A challenge in the field of nanoscience is to realize the full potential of self-assembled materials. To this end, the combination of biological, hierarchical self-assembly concepts with inorganic nano-particles that have well-defined physical properties is very promising. ‘Magnetic viruses’ (hybrid T7 bacteriophage) were generated which are uniform in geometry, physical properties, and biochemical functionality. After coaxing the DNA out of the T7 phage particles, magnetic particles were grown inside the remaining hollow viral capsid shells. Due to the benefits of phage display technology, this magnetic virus has the capability to integrate an affinity reagent against virtually any target molecule, which means that its bio-functionality can be precisely tailored. The approach provides an original method both to “tag” a bio-sample with nano-particles and to “bio-functionalize” a nano-particle. It differs from the conventional methods of fabricating bio-functional nano-particles, which involves the synthesis of nano-particle first with a subsequent anchoring (coating) of the bio-ftmctional ligands to the nano-particle surface. Instead, the current approach introduces a ligand integrated “bio-container”, which is then used as a template to fabricate the nano-particles. This approach can be further generalized to nano-particles with other desired physical properties, such as optical or radioactive, thus virus-templated nanoparticles will benefit the creation of nanomaterials in general and represents a unique method to approach major challenges in nanoscience and biology.

A T7 ghost was made by osmotically shocking a normal T7 phage. Since the density of encapsided T7 DNA (˜450 mg/mL) is at least 5-fold higher than that in metaphase chromatin, the DNA of T7 will burst to the outside after the T7 capsid is disrupted by the osmotic shock. By rapidly diluting pure T7 phages (10¹² pfu/mL) with a sodium sulfate solution (3M) in the presence of DNAase, it was found that the osmotic shock caused the escape of DNA debris (as a result of DNAase activity) from the capsid shell. Yet, the integrity of capsid shell remained after removing the osmotic shocking conditions. The shocked phage particles were collected by ultra-centrifuging (60,000 rpm for one hour at 4° C.). The ghost phages were then separated from the normal virus particles by banding in a cesium chloride density gradient (42% CsCl banding position for normal phage and 20.8% CsCl band position for the ghost phage), followed by dialyzing against the PBS buffer solution to remove cesium chloride. Capillary zone electrophoresis also confirmed the generation of T7 ghost phage. Based on the peak areas of the ghost particle and survived phage, it was estimated the yield of ghost phages generated by osmotic shock was 55%.

To visualize the ghost particles, phage samples prepared from the above methods were negatively stained with uranyl acetate (1%) and examined by Transmission Electron Microscopy (TEM, Philips CM-120). The packing of DNA inside a normal T7 phage is very tight; thus, preventing uranyl acetate presented at the virus core of intact viruses, as evidenced by a brighter contrast on the image. On the contrary, the ghost T7 particles are slightly shrunk, with an average diameter of 48 nm. Since uranyl acetate can diffuse inside the capsid, ghost particles have darker contrast in the middle . Most of ghost particles observed did not possess any tails, thus, it appeared that the tails became detached from the particles during the osmotic shock.

Europium-complex hybridized phage was synthesized by the reaction of europium ions with either naphthoyltrifluoroacetone (NTA) or dicarboxyic anthraquinone (DCAQ) at the presence of ghost T7 phage. Ghost T7s (0.4 mg/mL) were incubated with 4 mM of europium ions in an acetate acid buffer solution (pH=8.0) for one hour. 4 mM of NTA or DCAQ was then introduced and the resulting solution was incubated for three hours. During the incubation, insoluble europium-NTA or europium-DCAQ complex particles were formed. As the size of the particles increased, the diffusion of europium-complex particles inside the ghost phage were limited by the permeable size of capsid shell, thus larger europium-complex particles were put inside the ghost phage and continued to grow until the particles occupied the entire interior space of the ghost. The formed hybridized phage was purified by using magnetic beads coated with S-protein. Because of the specific interaction between the S-tag displayed T7 ghost and S-protein, the hybridized phages were immobilized on the surface of the beads. Europium-complex particles formed outside the phage were washed away with the acetate acid buffer solution containing 4 mM of NTA or DCAQ solution using a magnetic separator. Finally the hybridized phage was released from the beads using a competing assay by introducing T7 tag elution buffer (T7 Tag affinity purification Kit, Novagen).

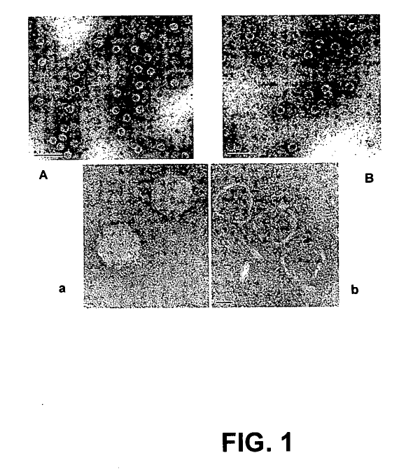

The formed europium complexed hybridized phage particles have a unique narrow fluorescence peak, centered at 675 nm, due to the presence of europium complex (FIG. 1A). Thus, the specific binding function of the hybridized phage against S-protein can be verified by a time-resolved fluorescence immunosorbent assay. An intact T7 phage, which went through the same procedures as making a europium complex hybridized phage, was used as a control. Both types of phages (hybridized and intact) were added to the microtiter plate wells coated with the S-protein and incubated at room temperature for 2 h. After the wells were washed three times with TBS, the fluorescence signals of the plate were recorded by a plate reader (Wallac 1420 Victor multilabel counter) equipped with a time-resolved fluorescence detector. As shown in FIG. 1B, a strong time-resolved fluorescence signal was observed in the sample of hybridized phage, indicating the strong binding between the S-protein and the hybridized phage. On the other hand, intact T7 phage only showed very weak signal because of the lack of europium complex. At the same time, a conventional ELISA, using 50 μL of S-protein labeled with horseradish peroxidase (HRP) and ABTS solution for visualization, was also conduced, and the ELISA confirmed the binding of both samples to the S-protein.

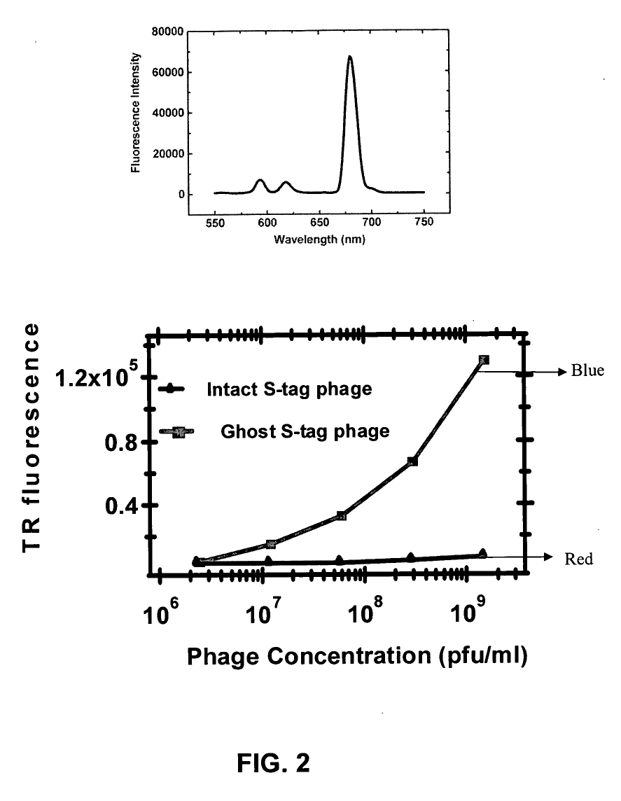

To visualize the hybridized phage particles, the samples were loaded onto a freshly glow discharged TEM grid and imaged directly without negatively staining. Hybridized T7 phages with uniform europium particles inside were dominated on observed TEM images. The size of europium-complex particles inside the ghost T7 is about ˜35nm in diameter with excellent mono-dispersed size distribution. EDX analysis confirmed the presence of europium (FIG. 2) as the major element inside the ghost T7.

Because the fabrication of europium-complex hybridizedT7 involves loading the cavity of T7 ghost with europium ions, a negatively charged phage capsid will help to attract Eu³⁺ ions, thus facilitating the synthesis of europium-complex particles inside the T7 ghost. An estimate of the exact interior charge nature of the T7 ghost is that the T7 capsid is negatively charged when it is in the solution of neutral pH (˜7) or above. This is supported by the fact that the capsid of T7 is made of many (up to eight) capsid proteins. The isoelectric point (pI) of major capsid protein (P10) is 6.0, and the pls of dominated internal protein (P15 and P16) of T7 capsid are 5.4 and 6.6, respectively. The negatively charged nature of the T7 ghost phage at neutral or base conditions benefits the loading of cationic europium ions, and so europium complex particles can be efficiently synthesized inside the T7 ghost.

The observed time-resolved fluorescence signal is almost 5 orders higher than the background noise in the time-resolved fluorescence immunosorbent assay experiments. Thus, the europium-complex hybridized phage probe provides a superior signal-to-noise ratio compared to the conventional fluorescent probes.

In summary, the generation of a europium-complex hybridized T7 phage presents a new class of fluorescent probes in which hybridized phages have mono-dispersed europium-complex particle core inside and affinity reagent displayed capsid shell outside. Hybridized phages, cobalt metal and rhenium oxide (an analog to ⁹⁹technetium oxide due to similar chemical properties of these two elements) have been synthesized. Because the inorganic cores in the hybridized phages are fully surrounded by the capsid proteins, in principle, the hybridized phage should be biocompatible in vivo. Hybridized phages are likely the next generation of probing reagents for bioassay, biosensor, targeted reagent delivery, and medical imaging.

The first step towards the synthesis of magnetic viruses was also to prepare ghost T7. Ghost T7 capsids have been obtained by normal release from host cells (very low yield) or by osmotic shock, which had a reasonably high yield of as high as 55%. Another procedure for preparing T7 ghosts described herein obtained yields as high as >98%. This was achieved by treating the purified T7 phage with an alkali buffer: the strong alkali condition denatures the capsid proteins and the DNA, permitting the DNA strand to escape. Followed by PEG precipitation of the phage, the capsids proteins are renatured, stable ghost particles form, and ghosts are collected by ultracentrifugation.

The high yield of T7 ghost particles was confirmed by transmission electron microscopy (TEM). In contrast to the normal T7, which have regular icosahedral heads (56 nm in diameter) and short tails (˜8 nm), the ghost T7 particles lose their symmetric nature, and are slightly shrunk with an average diameter of 48 nm. Both the normal and ghost T7 phage were negatively stained with uranyl acetate (1%) before TEM imaging. Since uranyl acetate can diffuse inside the capsid, ghost particles have dark core contrast, while the high packing density of DNA inside a normal T7 particle blocks the uranyl acetate uptake into the virus core of the intact viruses. Hence, the normal T7 phage particle has brighter core contrast.

The next goal was to use the ghost virus as a template to grow metallic Co inside the particle instead of the original DNA. This was achieved by utilizing the chemistry of reducing cobalt ions (II) with sodium borohydride. In brief, ghost T7 particles (0.4 mg/ml) were incubated with cobalt ions (II) in a degassed, buffered solution at room temperature. After a one-hour incubation, 10 mM NaBH₄ was added to the ghost T7-Co (II) mixture under N₂ over a two-hour period. A color change (pink to gray) indicated the formation of cobalt particles. Aside from using Co (II) ions, a control experiment was conducted with hexanitrocobaltate anions, which also can be reduced with NaBH₄ to form cobalt metal. However, the yield of magnetic cobalt viruses with hexanitrocobaltate is much lower compared to cobalt (II), which suggests that the negatively charged capsid proteins of the T7 ghost may play a role in attracting the positive cobalt ions, and suggests that positive metal ions are preferred for the formation of hybrid phage.

In any case, the cobalt particles form within the solution as well as inside the capsid, necessitating a purification step to isolate the magnetic viruses. This is accomplished by adhering the virus particles to a gold-coated substrate with a succinimidyl 3-(2-pyridyldithio) propionate (SPDP) cross-linker. The substrate is then subsequently washed to remove unbound linker, non-chemically bound phage and cobalt particles outside the ghost phage.

To visualize the magnetic viruses, the samples immobilized on a gold-coated TEM grid were imaged by TEM. Uniform cobalt particles (42±2 nm) were formed inside the ghost T7. This is supported by the energy dispersive X-ray (EDX) analysisFIG. 3, where the dominant element inside the T7 ghost was determined to be cobalt. The small particles (1-2 nm) in the background come from the gold particles used for anchoring the hybrid phage.

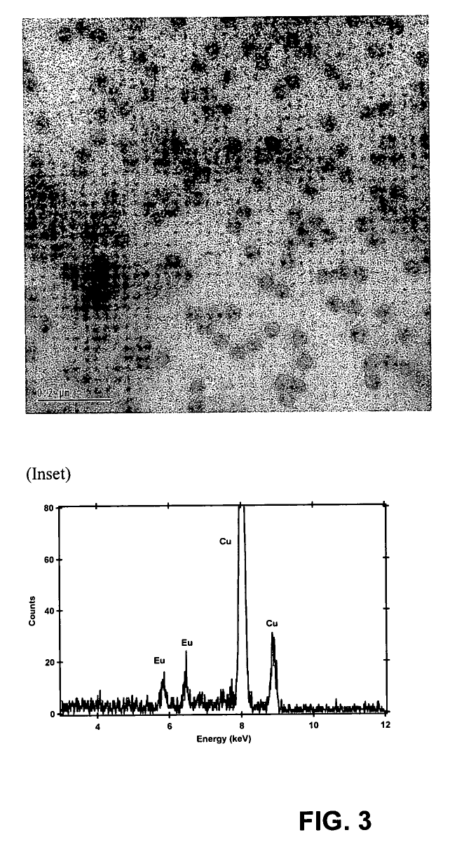

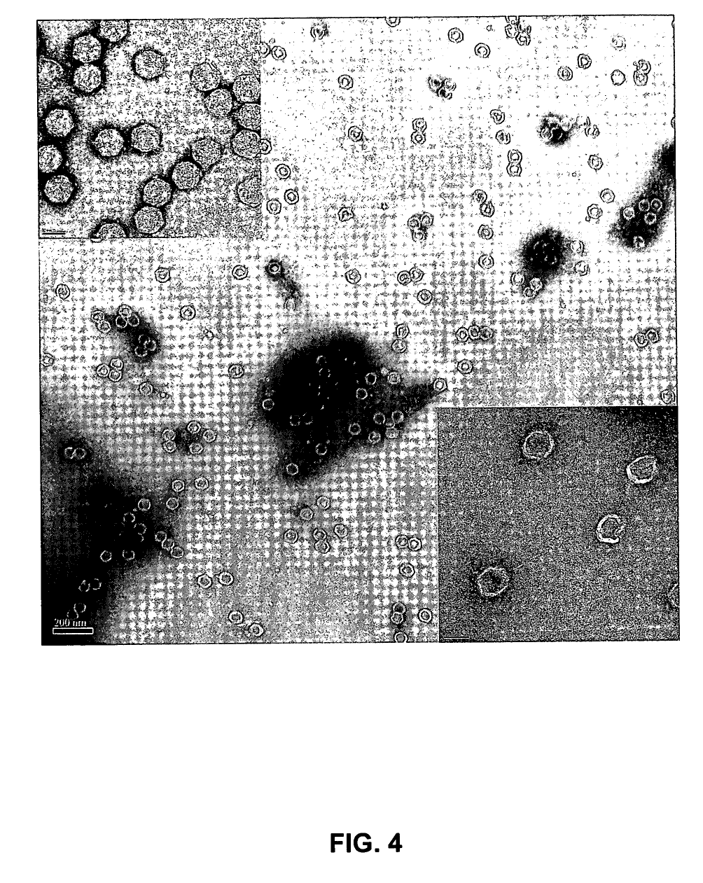

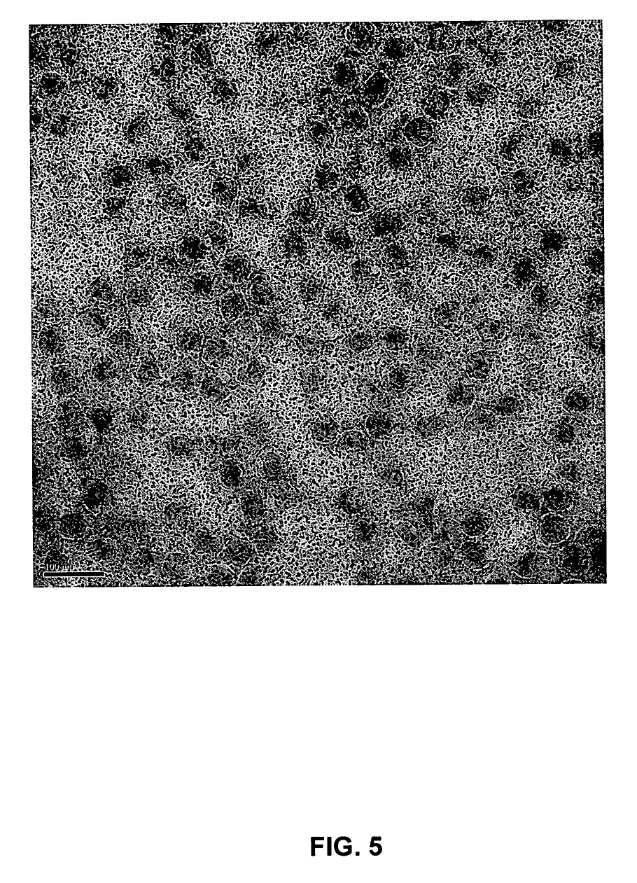

To determine if the bio-recognition capability of the capsid was preserved, Co viruses were prepared from wild type and S-tag peptide displayed T7 phage particles. Each S-tag peptide displayed T7 phage has 415 copies of a 15 amino acid (aa) S-tag displayed on its capsid and the S-tag peptide can interact with 104 aa S-proteins derived from pancreatic ribonuclease A with a 10 nanomolar dissociation constant. In contrast the wild type magnetic viruses serve as a negative control. The different binding affinity of the wild type and the S-tag displayed magnetic viruses was demonstrated using an enzyme linked assay. As shown in FIG. 3, when cobalt magnetic viruses bearing the S-peptide were added to microtiter plate wells coated with the S-protein, strong binding behavior was observed, whereas wild type cobalt magnetic viruses did not bind to the S-protein at all due to the lack of ligands on their surface. This behavior is the same as that known for the original viruses. Hence, ligand displayed magnetic viruses maintain their parent bio-recognition functionality.

The magnetic properties were then characterized. Measuring the room-temperature magnetization curve of cobalt magnetic viruses in liquid solution showed paramagnetic behavior, as indicated by the absence of hysteresis. This behavior is due to cobalt phages rotating freely in the liquid. In order to estimate the magnetic moment of each cobalt phage, the magnetization curve can be fitted to the classic model of paramagnetism (Langevin function), which is given by

$$M = {M}_{s} [ coth ( \frac{μ H}{{k}_{B} T} ) - \frac{{k}_{B} T}{μ H} ] ,$$

(1)

where u is the magnetic moment of each particle. From this fit the magnetic moment for each cobalt phage is 4.7×10⁴ μ_{B}, which is smaller than the theoretical value 5.2×10⁶ μ_{B} estimated for a spherical single-domain cobalt nanoparticle with 40-nm diameter using the value of the bulk saturation magnetization of cobalt (Mₛ=1440 emu/cm³) at room temperature. This reduced magnetization could be either due to multiple ferromagnetic domains, which could reduce the net magnetization, or due to oxidation or hydration of the cobalt during or after the precipitation process. Below the freezing point of the liquid, the magnetization shows hysteresis with non-zero coercivity as expected for ferromagnetic particles.

In order to ascertain that an individual cobalt phage is truly ferromagnetic at room temperature, the magnetic behavior of cobalt-hybrid phage that was immobilized on a gold-coated substrate was measured. The coercivity for these immobilized cobalt phage decreases with increasing temperature, but remains non-zero at room temperature indicating that the magnetic viruses are truly ferromagnetic. Therefore, the paramagnetic response of the phage suspended in the liquid is indeed due to rotational Brownian diffusion of the whole particle.

The magnetic viruses are a new functional entity, for example in biosensing applications. The frequency response of the magnetic susceptibility could be used as follows: the imaginary part of the magnetic susceptibility X has a peak when the applied frequency is the inverse of the Brownian relaxation time,

$${τ}_{B} = \frac{π η {d}^{3}}{2 {k}_{B} T} ,$$

(2)

where η is the viscosity of the liquid, and d is the particle's effective hydrodynamic diameter. S-protein attachment to suitable coated magnetic nanoparticles was detected, because the hydrodynamic diameter and hence the relaxation time increases after binding. This substrate-less approach allows for easy mix-and-measure sensing, where the analyte is mixed into the solution of ferromagnetic particles without any need for further sample preparation. The magnetic virus, with its sharp size control, is expected to yield sharp resonant peaks in χ″ that make the frequency shift analysis an even more powerful tool. Magnetic susceptibility measurements of the cobalt magnetic viruses in aqueous solution show a peak in χ″ at ˜5 kHz. The estimated hydrodynamic diameter is 70 nm using the dynamic viscosity of water at 280 K. This peak disappears when the solution is cooled to 250 K, because the freezing of the liquid immobilizes the nanoparticles. This implies that the frequency peak at room temperature is due to the rotational diffusive Brownian relaxation of the magnetization and thus the magnetic viruses are suitable for biosensing applications. In contrast to the previously used magnetic nanoparticles, the availability of almost any affinity reagent through phage display allows a straight-forward adaptation of this sensing approach to virtually any target using the magnetic viruses.

Developing efficient methods to fabricate and organize nanometer scale building blocks into functional architectures that extend over microscopic and macroscopic length scales is the key to the realization of magnetic nanotechnologies. Nature produces complex multi-ftunctional objects through the self-assembly of widely disparate molecular constituents organized over many different length scales. Viruses, in this regard, exemplify an extraordinarily organized nano-architecture: They are complex molecular biosystems in which nucleic acid strands are confined within a nano-sized compartment (capsid), which ranges from 20-100 nm; depending on the virus. The simplest viruses are formed through a self-assembly process; more complex viruses assemble through processes that may require non-structural proteins coded either by the virus or host cell. The function of the capsid is to provide a rigid container protecting the viral nucleic acids during the passage from one host cell to another. From a material science point of view, the organized biological architecture provides a spatially-defined host system which can be used as a template for uniform fabrication of structured materials with different length scales. For example, bacterial S-layers have been used as a defined entity to accommodate the formation of polymer material in micrometer scale for drug delivery lipid assemblies, DNA and multicellular superstructures have been used to direct the patterning and deposition of inorganic material in micro- to nanometer scale. The use of the phage capsid as the template for materials fabrication challenges the traditional view that materials synthesis is confined to the thermodynamics of condensed matter. Instead, it offers the new approach of “synthesis by design” and a revolutionary way to “tag” or “functionalize” the magnetic nano-particles. Packaging of magnetic material within a phage particle can result in formation of a highly uniform population of protein-encased magnetic particles. Furthermore, phage coats can be modified to incorporate binding sites with both high affinity and high specificity to a particular molecular target (such as a bacterial spore). Consequently, the use of viral assembly as a mode of materials fabrication has advantages difficult to achieve in the absence of biological materials.

For the applications envisioned here, T7 phage are preferable to bacteriophage M13 in displaying protein segments because their capsid proteins will accept C-terminal fusion. Libraries in this bacteriophage, which contain cDNA inserts and display their encoded proteins on their surface, can be screened by affinity selection, as described above for bacteriophage M13. For example, it is possible to screen a library of T7 phage displaying proteins encoded by cDNAs, with biotinylated RNA segments, corresponding to RNA regulatory elements, and specifically affinity select RNA-binding proteins. Novagen (Madison, Wis.) offers libraries of T7 phage displaying cDNA segments corresponding to mRNAs of HeLa cells, and human stomach, liver, and breast cancer cells. Fragments of antibodies, single-chain fragments of variable regions (scFv), were displayed on the surface of T7. A library is contemplated from which antibodies are selected that bind to target proteins of interest. Thus, phage-displayed cDNA and antibody libraries have the potential to identify candidate interacting proteins and antibodies, respectively, for many proteins of interest.

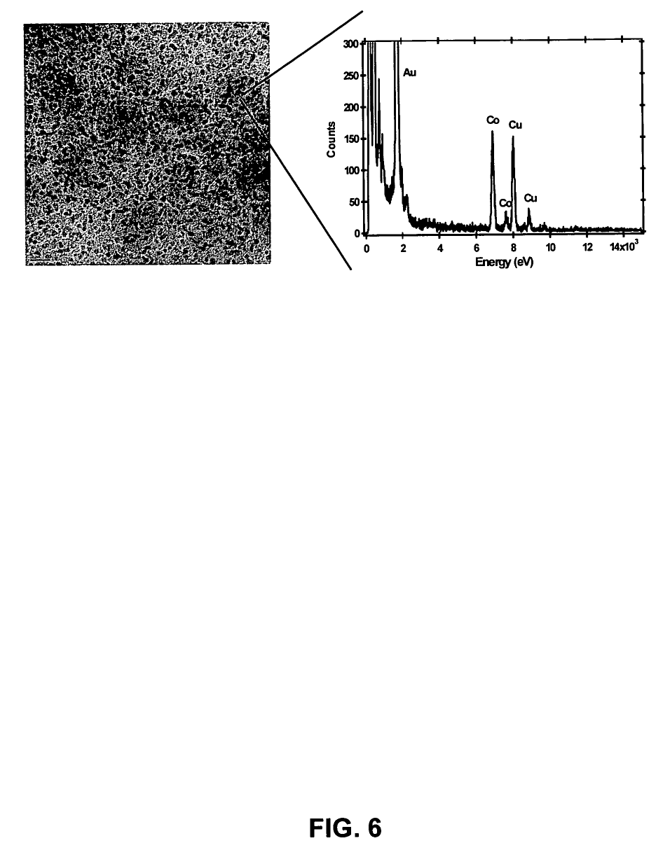

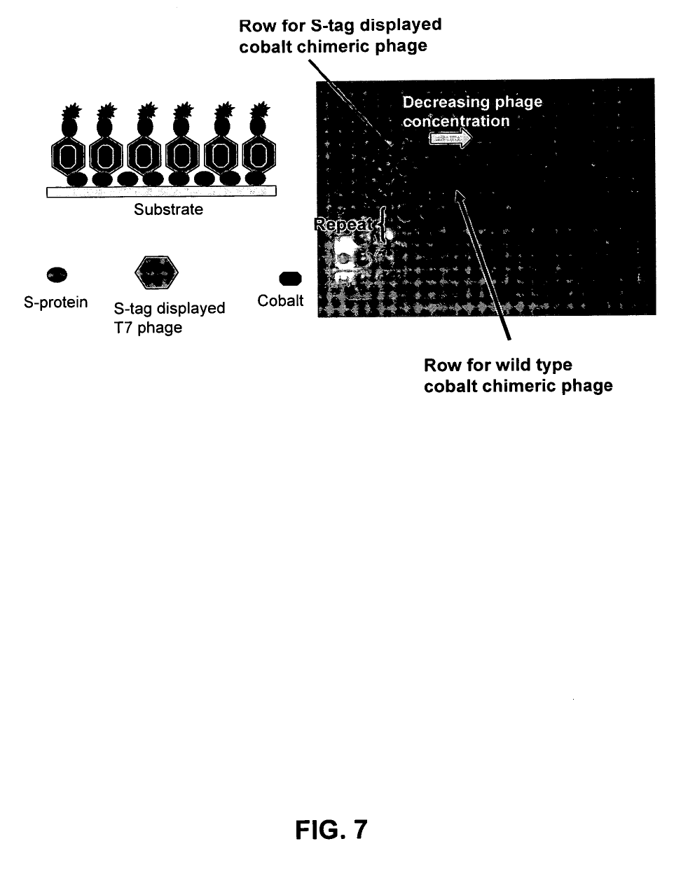

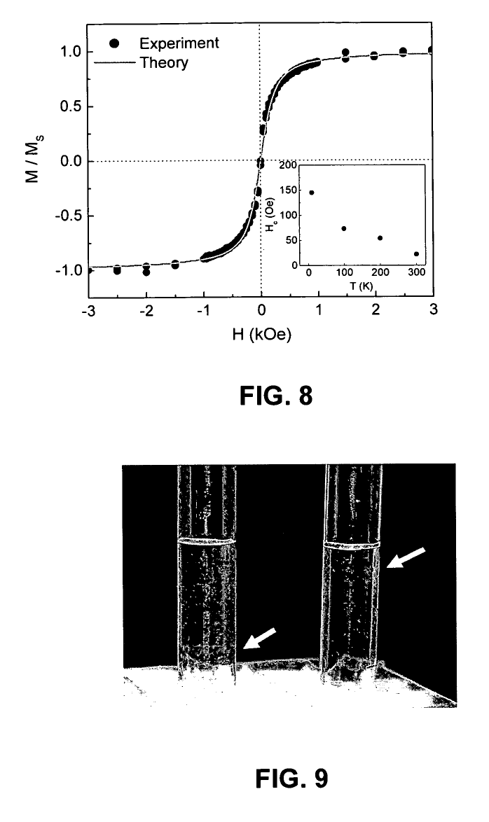

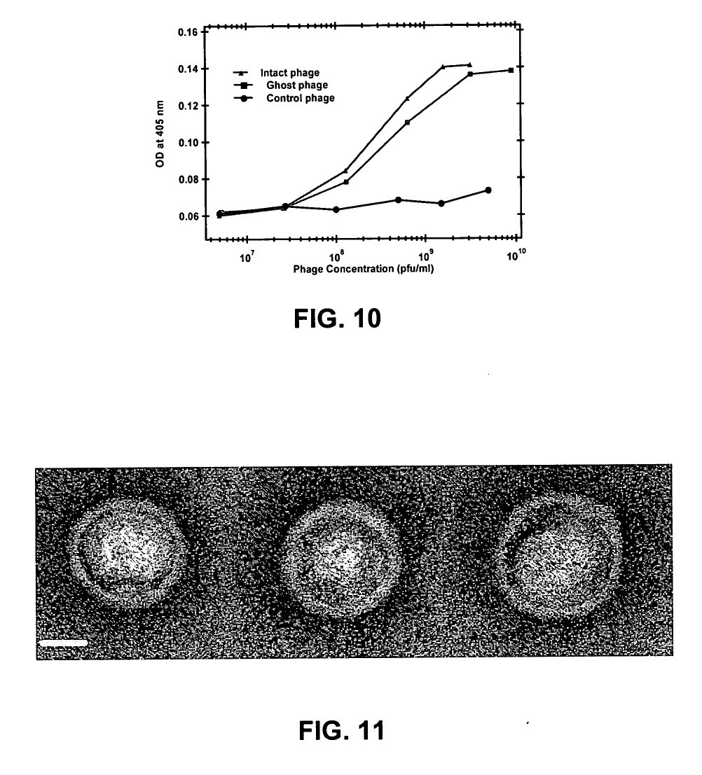

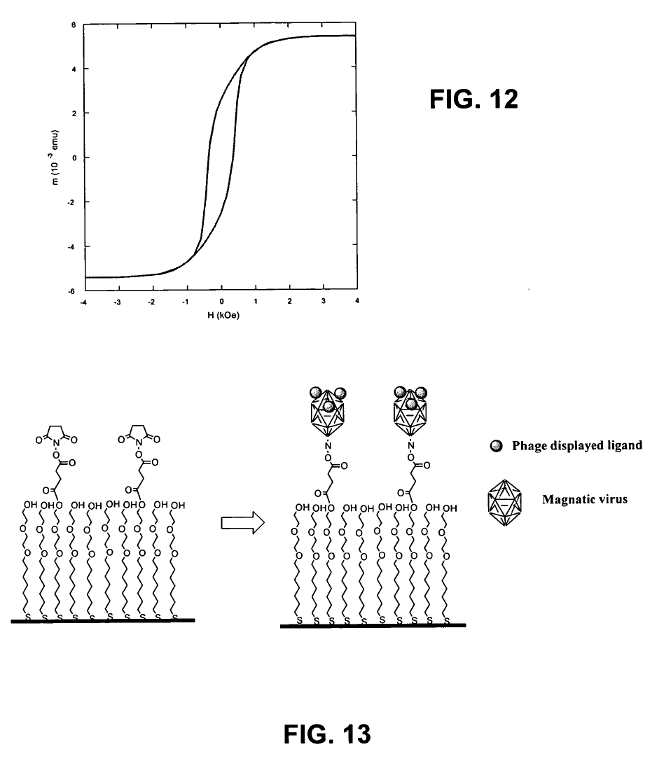

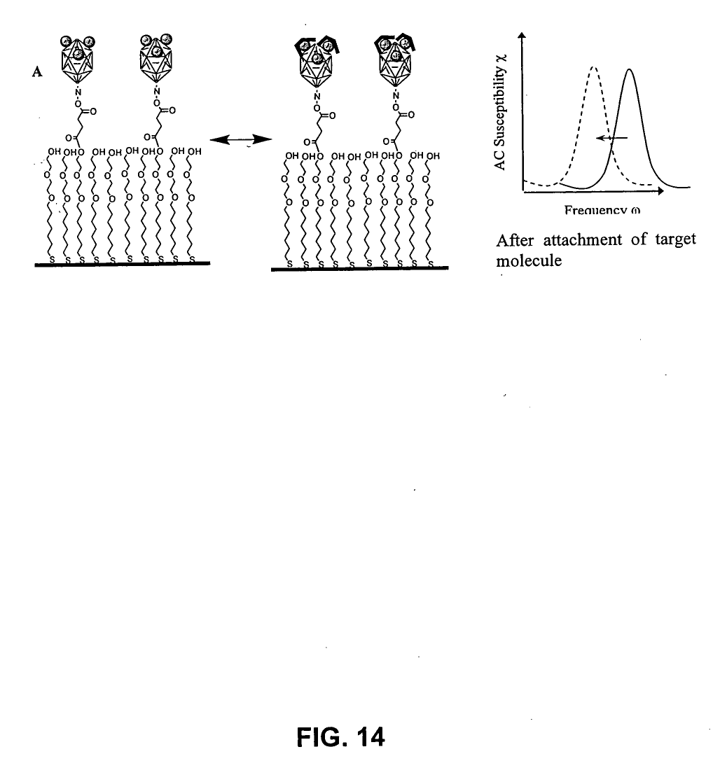

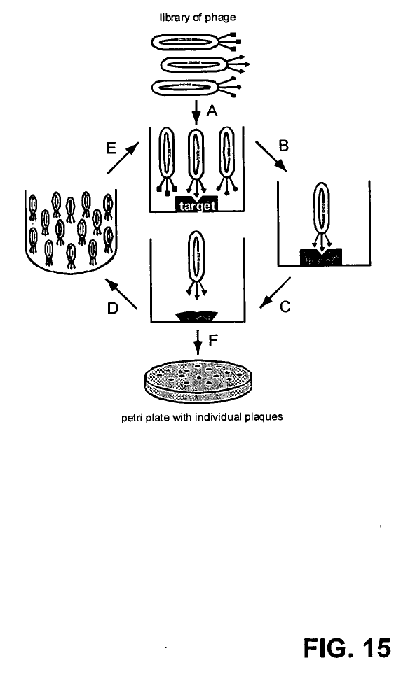

Magnetic nano-particles are suspended in aqueous solution and the nanoparticle is integrated into a nano-mechanical oscillator. This is accomplished by binding the magnetic nano-particle to a substrate through a flexible linker (See FIGS. 6-8). This provides a mechanism to extend the idea of biosensing through changes of dynamic magnetic properties down to the single molecule detection limit.

Magnetic material is enclosed within a viral capsid that exhibits selective binding to a protein, providing a molecular basis for highly sensitive monitoring of the presence of target protein by measuring the resonant oscillating frequency of the magnetic viruses.

Empty T7 bacteriophage were used as the template for the fabrication of magnetic particles. T7 is a fast growing and extremely stable double strand DNA phage. Since peptides or proteins displayed on the surface of T7 do not need to be capable of export through the periplasm and the cell membrane, T7 offers a great advantage compared to filamentous phage in terms of construction of the phage display library. For example, up to 10 copies of peptide and larger proteins up to 1200 amino acids can be displayed on the T7 phage surface, while peptides up to 50 amino acids can be displayed in a number of 415 copies per phage. Bacteriophage M13 can only tolerate short peptides or protein fragment on its surface. In addition, the conformation of T7 is superior for providing a confined template for material synthesis. The structure of the T7 phage particle includes the capsid shell, head-tail connector, tail and tail fibers. The diameter of the icosahedral phage head is in the order of 55 nm, thus a T7 can provide a cavity of 40 nm after devoid of its DNA; in contrast the M13 viral particle is 9 nm wide and 1 micro long, and can not be made as ghosts. To incorporate specific bio-recognition processes into the T7 phage particle, a special T7 phage particle was used, on which 45 copies of a 15 amino acid (aa) S-tag peptide were displayed (wild type of T7 phage particles can be used as the negative control). Thus, through this peptide-protein interaction, affinity purification was achieved for the phage particles, as well as their derivatives.

Phage particles were negatively stained with uranyl acetate and examined by Transmission Electron Microscopy (TEM). Since uranyl acetate can diffuse inside the capsid, ghost particles have a dark contrast in their middle. Normal T7-phages have icosahedral phage heads of 56 nm in diameter and short tails less than 8 nm. The packing of DNA inside a capsid is very tight, thus no uranyl ions are evident in the virus core of intact virus particles. The ghost T7 particles are slightly shrunk with an average diameter of 48 nm. No tailed phage were observed among the ghost particle, possibly because the tails became detached from the particles during the osmotic shock.

However, 90% of ghost particles generated from osmotic shock remained encapsulated structures. Encapsulated ghost phages are essential for templated synthesis of magnetic particles since the confined interior can host the synthesis reaction. The generation of T7 ghost phage is also confirmed by capillary zone electrophoresis. The separation of ghost phage with wild type was carried out in an untreated fused-silica capillary (50 mm inner diameter, 50 cm length,) at 25° C. and monitored with UV detection. The separation buffer was 100 mmol borate (pH 8.5) containing a small amount of detergents to prevent viral aggregation and absorption to the capillary wall. A retention time of 4.5 min was assigned to the wild type phage, while the peak at 5.3 min was due to the presence of ghost particles in the solution. Based on these experimental results, a conclusion is that using sodium sulfate osmotic shock provides an efficient way for generating ghost particles suitable as templates.

To demonstrate that the peptides or proteins displayed on the surface of ghost particles retain their binding functions, an enzyme linked immunosorbent assay (ELISA) was used. When phage particles bearing the S-peptide were added to microtiter plates wells coated with the S-protein, equivalent binding was observed for both the intact and ghost particles, while wild type control phages did not bind to the S-protein at all due to the lack of ligands on their surface. This positive result supports that ghost phages can display different ligands on their surface.

Indeed, the ghost T7 synthesized provides an excellent template for the further synthesis of iron oxide nano-particles. After incubating the ghost phage (0.4 mg/ml) with 20 mM of Fe(NH₄)₂SO₄ in pH=5.0 buffer solution in the air, Fe (II) were distributed both inside and outside of the ghost phage. Subsequently, Fe(NH₄)₂SO₄ was oxidized by oxygen to yield iron oxide particles. The outside iron oxide particles can be easily separated by the centrifuge due to their relative large size. The supernatant which contains ghost phages was stained by uranyl acetate and imaged by TEM. Particles were formed inside the ghost phage. Compared to the hollow ghost, which contains uranyl acetate, and thus has darker contrast inside the phage, the ghost phage observed here is occupied by the formed particle, which prevents the diffusion of uranyl acetate into the phage, and thus only shows darker contrast rings along the phage shell. After the dialysis against a PBS buffer, the formed particles inside the phage were confirmed to be iron oxide from the absorption peaks at 540 nm.

Magnetic particles with sizes similar to the capsid of T7 phage are viable for room-temperature magnetic signal transduction. Magnetic characterization of magnetite nanoparticles, which were biomineralized by Magnetospirillum magnetotacticum bacteria was performed. These magnetite nanocrystals have a typical size of 42 nm in diameter, and are thus of comparable size as the magnetic core expected for the magnetic virus based on ghost T7. A typical magnetic hysteresis taken at room temperature has a rounded hysteresis loop and reduced remanent magnetization, due to the random orientation of the crystalline anisotropy from different orientations of individual nanoparticles in this measurement. The particles are well removed from the superparamagnetic region, which is reflected in the fact that there is only a very small increase (about 10%) in the saturation magnetization upon cooling down to 10 K.

The production of narrowly dispersed, nanometer-sized particles remains a significant challenge in material science. The critical difficulty is that these particles tend to aggregate and grow in order to minimize the overall surface free energy. Therefore, free precipitation is often not a viable technique. As discussed in the background section, biological systems, which have precisely confined domains, provide a unique template for the material fabrication. Domains can serve as the barriers to eliminate the particle aggregation. The principle of precipitation in a highly constrained bio-compartment can be understood as the following: assuming the aqueous core and particle are spherical, and all encapsulated precursors will form the target particle, the particle diameter upon precursor exhaustion is described by the equation of d=D(M/Mw/p)^{1/3}, where d and D are particle and container diameters, M the internal precursor molarity, Mw the molecular weight of the product, and p the product density (in g/cm³). Therefore, particle sizes can be manipulated by varying the solution concentration and container sizes.

The generation of encapsulated empty T7 phage by Na₂SO₄ osmotic shock and the fabrication of iron oxide within the phage virus was achieved. Magnetite (Fe₃O₄) nanoparticles are fabricated inside the T7 capsid. Magnetite has the advantages of having a Curie temperature (T_{c}=585° C.) well above room temperature, being chemically stable, and compatible with biological environments. Magnetite nanoparticles with diameter >10 nm, the minimum size necessary to avoid superparamagnetism at room temperature, which for magnetite is ˜7 nm in diameter. Typically, magnetite nanoparticles are prepared by co-precipitating Fe²⁺ and Fe³⁺ ions in an ammonia or sodium hydroxide solution. Ghost T7 phage is stable in the presence of OH⁻ (up to pH=12). Thus, the magnetite is fabricated using standard aqueous precipitation techniques in situ inside the capsid. Furthermore, magnetic properties of these nanoparticles are tailored by using magnetite related ferrite particles (such as CoFe₂O₄), which can be prepared in a similar manner. The final purification of magnetic phage is achieved by either affinity chromatography or capillary electrophoresis.

In order to produce a high yield of capsid encapsulated magnetic particles, the issue of compatibility of the interior of T7 ghost and the starting material for the synthesis of magnetic particles is addressed. Under normal conditions, the native T7 viral capsid has a cationic interior since the cationic nature favors hosting the polyanionic DNA. Since the synthesis of magnetite particles inside T7 ghost is altered involves the encapsulation of cationic species Fe II/ Fe III, the charge nature of the interior T7 ghost is altered to anionic so that it can provide a complementary electrostatic interaction with Fe II/Fe III. This goal of changing the chemical and physical properties of the interior surface of the capsid without disrupting the overall virus architecture can be accomplished by displaying negatively charged peptide or proteins on the surface of interior capsid. For example, some basic residues at the N-terminal or C-terminal of capsid protein 9 (inner shell capsid protein of T7) are replaced with glutamic acids. Conditions have to be used so that such a modification does not destroy the viral structures. For example, the charge nature of the capsid cavity may be inverted to facilitate the loading of Fe II and Fe III species. Even in the case that such a modification disturbs the packing of DNA of T7 during the infection, the formed capsid still can serve as the template.

Since the size distribution of the magnetite is important for the function of the proposed devices, capsids are produced with different sizes of mutated scaffolding proteins attached. In this case, scaffolding proteins serve as an inner layer of the viral shell, and reduce the effective inner-diameter of the capsid. Magnetite particles with sizes spanning from a few to 50 nanometers (the maximum size that can be accommodated within a T7 capsid) can be generated inside the ghost virus.

The size and morphology distribution of magnetite phage can be characterized by electron microscopy. The core magnetic particles can be examined by atomic force microscopy after the decomposition of shell proteins. The magnetic virus also can be characterized by compositional mapping using spatially resolved x-ray fluorescence spectroscopy, a unique resource of Argonne's Advanced Photon Source synchrotron, as well as magnetic structure characterization with small angle neutron scattering available at the Intense Pulsed Neutron Source at Argonne.

Materials and Methods

Preparation, Isolation and Purification of T7 Virus and its Corresponding Ghost Virus

Both wild-type and S-tag displayed bacteriophageT7 (Novegen) were grown on E. Coli (BL-21) using LB medium (Lennox L broth). After lysis of cells, the bacteriophage particles were precipitated by use of polyethylene glycol and purified by the use of centrifugations in cesium chloride step density gradients. In brief, a single colony of E. Coli Bl-21 was used to inoculate 50 ml of sterile LB broth, followed by shaking the solution at 250 rpm overnight at 37° C. 10 ml of the above culture was then diluted 100 times using fresh M9LB medium. The culture was shaken at 250 rpm at 37° C. until the O.D. of the culture at 600 nm reaches 0.8. Infection of the culture was achieved by adding 100 μl stocked S-tag displayed T7 phage. The mixture solution was kept shaking until cells were lysed (the solution became clear). 115 ml of 5 M NaCl was then added into the flask and the solution was incubated on ice for 20 minutes. Cell debris was removed by spinning the lysis at 9000 RPM for 20 minutes. The supernatant was treated with 1/6 volume of 50% PEG, stirred 4° C. for overnight, followed by standing on ice for 30 minutes.

The solution was spun at 8000 rpm for 15 minutes at 4° C. The phage pellet was then re-suspended in 15 ml PBS. Further purification was achieved by loading PEG-extracted phage onto CsCl step gradients premade in Beckman 344059 centrifuge tubes, as described in the Novagen T7select system manual, and centrifuging at 35,000 rpm for 60 minutes at 4° C. using a Beckman SW41Ti rotor. The purified phage was collected by extracting the phage band, followed by dialysising against PBS to remove CsCl.

To make to the ghost of native and S-tag displayed T7 phages, phage particles in lml of the above phage solution was spun down at 60,000 rpm for 60 minutes using a Beckman TLA 100.3 rotor. The phage pellet was then treated with 0.5 ml alkali buffer (0.1 M NaOH, 10 mM EDTA), and incubated at room temperature for 10 minutes. 25 ml PBS, 1/10 volume of 5 M NaCl and 1/6 volume of 50%PEG were then immediately added, and the solution was placed on ice for 2 hours. The ghost particles were spun down at 10,000 rpm for 20 minutes at 4 ° C. The ghost pellet was then re-suspended in 3 ml PBS in 3 ml of saline (150 mM NaCl). The purified ghost phage was collected by spinning down the above solution at 60,000 rpm for 60 minutes, followed by re-suspending the ghost phage in 50 μl 150 mM NaCl.

Cobalt Metal Hybrid Phage Particles

The stock of NaCl/MgSO₄ (150 mM) buffer solution was degassed by bubbling the solution with nitrogen for 2 hours. Stocks of 200 mM cobalt acetate or sodium hexanitrocobaltate solution and 100 mM sodium borohydride were prepared using the above buffer and further degassed for another hour. 5 μl cobalt ion solution was mixed with 100 μl of the ghost stock solution, and the mixture was incubated at room temperature for one hour. Reduction of the cobalt ion was carried out by gradually adding 10 μl sodium hexanitrocobaltate solution under the nitrogen. For the best results, an addition rate of 1 μl per minute was used. The solution was stirred for two hours at room temperature. Aliquots were taken and used for further purification and TEM imaging.

ELISA

The binding activities of hybrid phage were characterized by an enzyme linked immunosorbant assay (ELISA). In brief, 100 microliter of diluted S-protein (10 microgram/ml) were aliquoted into 96-well microtiter plates, incubated at room temperature for 3-4 hour or at 4° C. overnight, followed by extensive washings with 300 microliter 1×TBS. After washing away unbound proteins, each well was covered with BSA blocking reagent. Purified T7 phages, ghost T7 particles or hybrid phages (50 ul, serially diluted five-fold in PBS) were added to wells containing S-protein and block reagent and incubated at room temperature for 2 hours. Consequently, solutions of S-protein labeled with horseradish peroxidase (HRP) (50 ul) were added to each wells for 1 hour at room temperature, and visualized by adding 50 microliter of freshly prepared ABTS solution (2,2′-azinobis(3-ethylbenthiazoline-6-sulfonic acid, 0.22 mg/ml solution of 386:614 (v/v) mixture of 0.2 M Na₂HPO₄ and 0.1 M citric acid, as well as 1/1000 vol of 30%(w/V) H₂O₂). The wells are then allowed to react for one hour at room temperature before being read on a plate reader (Wallac 1420 Victor multilabel counter). Binding activities were quantified by O.D. at 405 nm.

Transmission Electron Microscopy Analysis

Sample preparation: The samples were supported on 400-mesh carbon coated grids, freshly glow discharged (Evaporator: EDWARDS AUTO 306) for 45 second. The specimens were negatively stained by first applying a drop (5 ul) of phage or ghost phage to a grid. The grid was then washed with water, and stained for 30 second with aqueous solution of uranyl acetate (1%) (phage and ghost phage samples), then wicked off with filter paper and allowed to dry. For the cobalt-hybrid phage sample, the TEM grid was coated with a layer of gold particles (2 nm in thickness) using a thermal evaporator (Denton Vacuum) via a deposition rate of 0.5 nm/sec for 2 second. The gold coated grid was then modified with protein cross-linker by immersing the grid into 10⁻⁵M succinimidyl 3-(2-pyridyldithio) propionate in DMF solution overnight at room temperature, followed by extensive washing steps to removing any bound SPDP. The hybrid phage was immobilized to the grid surface by covering the SPDP modified substrate with the aliquot of cobalt hybrid phage for one hour. After extensively washing the grid with pure water, the sample was imaged without negative staining.

Method: Electronic microscopic images were obtained from a FEI Tecnai G2 F30 transmission electron microscope quipped with an Oxford EDX analyzer. The microscope was operated at 300 kV. Micrographs were recorded on a Gatan CCD digital camera.

Magnetic Characterization of Cobalt Hybridphage

For the magnetic characterization, a 100-μl aliquot of the solution with 10¹²-10¹³ cobalt phages was used. The magnetization of the samples at various applied magnetic fields and temperatures were measured in a superconducting quantum interference device (SQUID) magnetometer, and ac magnetic susceptibilities were measured in a Physical Property Measurement System (Quantum Design, San Diego, Calif.). An ac amplitude of 10 Oe was applied for all the ac measurements, while the frequency was varied between 10 Hz and 10 kHz.

Instruments

Absorption spectra were recorded on a Hewlett-Packard 8453 diode array spectrophotometer. A Wallac 1420 Victor multilabel counter (Wallac Inc.) was used to read the ELISA plates. The protein concentration was characterized by using a ND-1000 spectrophotometer (Nano Drop Technologies, Inc.).

Preparation of T7 phage and the corresponding ghost phage

Both wild-type and S-tag displayed bacteriophageT7 (Novagen) were grown by infecting mid-late log phase E. coli (BL-21) cells by using LB medium (Lennox L broth). After lysis of cells, the bacteriophage particles were precipitated by using polyethylene glycol and purified by using centrifugations in cesium chloride step-density gradients. Further details are provided by Novagen's protocols.

Ghost T7s were prepared by osmotic shock of purified T7 phage with 2M Na₂SO₄ solution. After the escape of DNA from phage particles, the formed ghost phages were collected by using refrigerated centrifuge and purified by banding in a cesium chloride density gradient. In brief, 1 mL of purified phage stock was added to 2 mL of 3M Na₂SO₄, which had been pre-warmed at 37° C. The suspension was shaken for 2 min at 37° C and poured into 50 mL of 4° C. distilled water. The solution was stirred vigorously overnight at 4° C. and then centrifuged at 60,000 rpm (100,000 g) for 1 h at 4° C. (Beckman TLA-100.2 rotor). After all of the supernatant was removed, the pellets were dissolved and pooled into a total volume of 5 mL saline. T7 ghost particles were isolated by CsCl step-gradient centrifugation at 4° C. for 1 h at 35,000 rpm (Beckman SW 41 rotor). After centrifugation, a thick layer of empty phage heads (ghosts) formed on top of the 20.8% CsCl layer. The CsCl-s purified ghost particle fraction was collected and dialyzed against saline at 4° C. overnight, and the saline solution was changed three times during this period

Hybridized Phage Particles

Ghost T7 particles (109 pfu/mL) were incubated with 4 mM of EuCl₃ in acetate acid buffer solution (pH=8.0) for 30 min. 4 mM of trifluoroacetyl acetyl naphthlebe (TAN) or dicarboxyic anthernthed quesquo (DCAQ) was then introduced into the solution. The mixture was incubated at 4° C. for 2-4 h. The hybridized phage was purified by using magnetic beads (BioMag Magnetic immobilization Kit, Bangs Lab, Inc.) coated with anti-T7 antibody (T7 Tag affinity purification Kit, Novagen). Because of the specific interaction between the T7 ghost and anti-T7 antibody, the ghost phage binds to the beads. After the beads were washed three times with the acetate acid buffer solution containing 4 mM of TAN or DCAQ, the hybridized phage was released from the beads by using T7 tag elution buffer. The magnetic beads were removed by centrifuge (8,000 rpm for 5 min), and the hybridized phage was finally collected by using an ultra-centrifuge (60,000 rpm for 30 mins) at 4° C.

ELISA

The binding activities of phage particles were characterized by a time-resolved fluorescence assay and an enzyme linked immunosorbant assay (ELISA). In brief, 100 μL of diluted S-protein (10 μg/mL) was aliquoted into 96-well microtiter plates, incubated at room temperature for 3-4 h (or at 4° C. overnight), and then washed extensively with 300 μL 1×TBS. After unbound proteins were washed away, each well was covered with BSA blocking reagent. Purified T7 phage, ghost T7 particles, or hybrid phages (50 μL, serially diluted five-fold in PBS) were added to wells containing S-protein and block reagent and incubated at room temperature for 2 h. After the wells were washed three times with TBS, the fluorescence signals of the plate were recorded by a plate reader (Wallac 1420 Victor multilabel counter) equipped with a time-resolved fluorescence detector. Binding activities were quantified by using a program specifically designed to be used for analyzing the europium complex. To confirm the binding of phage particles, solutions of S-protein labeled with horseradish peroxidase (HRP) (50 μL) were also added to each well for 1 h at room temperature and visualized by adding 50 μL of freshly prepared ABTS solution (2,2′-azinobis[3-ethylbenthiazoline]-6-sulfonic acid, 0.22 mg/mL solution of 386:614 [v/v] mixture of 0.2 M Na₂HPO₄ and 0.1 M citric acid, as well as 1/1000 vol of 30%[w/V] H₂O₂). The wells are then allowed to react for 1 h at room temperature before being read on a plate reader (Wallac 1420 Victor multilabel counter). Binding activities were quantified by O.D. [Optical Density] at 405 nm.

Instruments

Electronic microscopic images were obtained from a Philips CM-120 transmission electron microscope quipped with an oxford EDX analyzer. The samples were supported on 400 mesh carbon-coated grids, freshly glow discharged (Evaporator: EDWARDS AUTO 306) for 45 s. Specimens were negatively stained by applying a drop (5 μL) of phage particles to the grid. The grid was then washed with water and stained for 30 s with an aqueous solution of uranyl acetate (1%), and then the solution was wicked off with filter paper and allowed to dry.

The microscope was operated at 120 kV. Micrographs were recorded on a Gatan CCD digital camera.

The separation of ghost phage with wild type was carried out in a backman PACE Capillary Electrophoresis System 5000 using an untreated fused-silica capillary (50 mm inner diameter, 50 cm length,) at 25° C. and monitored with a UV detector. The separation buffer was 100 mM borate (pH 8.5) containing a small amount of detergent (0.1 % Tween 20) to prevent viral aggregation and absorption to the capillary wall. A peak with retention time of 4.5 min was observed when the sample from the upper layer of the cesium chloride density gradient (normal T7 phage) was used. The peak shifted to 5.3 min when the sample from the lower layer of the cesium chloride density gradient was used because ghost particles were in the solution.

Absorption spectra were recorded on a Hewlett-Packard 8453 diode array spectrophotometer. A Wallac 1420 Victor multilabel counter with a time-resolved fluorescence detector (Wallac, Inc.) was used to read the ELISA plates. The protein concentration was characterized by using a ND-1000 spectrophotometer (Nano Drop Technologies, Inc.).

Screening of Libraries of Phage-Displayed Virons:

Phage libraries are screened to identify specific phage against targets with potential for use as biowarfare agents. The initial target will be B. subtilis spores because of their similarity to B. anthrancis spores. To identify T7 phage particles that bind to target proteins (coat protein CotE) on spore surfaces, both cDNA and antibody fragment libraries will be screened by affinity selections. Novagen (Madison, WI) offers libraries of T7 phage displaying cDNA segments corresponding to mRNAs of HeLa cells, and human stomach, liver, and breast cancer cells. Inserts, encoding scFv, from a bacteriophage M13 library, are transferred into T7 phage vectors. With four different antibodies, scFv's can be functionally displayed on the surface of T7 virus particles. Aliquots of both libraries are after three rounds of selection, individual clones will be grown up and tested by ELISA for binding to target coated microtiter plate wells. Target-binding phage will then be characterized by DNA sequencing. Subsequently, the corresponding magnetic phage can be synthesized via similar approach as previously mentioned.

2D Assembly of “Magnetic Virus”:

2D arrays of single-domain magnetic virus via self-assembly monolayers (SAM) are assembled on a gold surface. Tri-ethylene glycol substituted alkanethiol is synthesized from commercially available hexadecanediol, and further converted to the succinimidyl derivative. Consequently, the SAMs can be prepared by immersing gold coated substrates in an ethanolic solution containing the mixture of tri-ethylene glycol and succinimidyl substituted alkanethiols. After the substrates are rinsed with ethanol and dried with nitrogen, the magnetic phage particles can be attached the SAMs in a phosphate buffered saline solution containing 10 mM of sodium hydro carbonate due to the reaction of amines in the capsid proteins of T7 with succinimidyl group. The anchoring of magnetic phage to the SAMs can be confirmed by Atomic Force Microscopy (AFM), surface plasma resonance spectroscopy, or x-ray and neutron reflectometry. Quantitative characterization of the assembly can be achieved by incubating the substrate with fluorescence dye labeled S-protein. Due to the specific interaction of S-protein with magnetic phage, the amount of magnetic phage attached to the SAMs can be quantified by fluorescence spectroscopy based on the emission of labeled S-protein.

ac-Susceptibility Measurements

Measurements with ferromagnetic viruses in solution are made, similar to the detection technique proposed by Connolly and St. Pierre [J. Connolly et al., 2001]. First, measure the magnetic ac susceptibilities of the “original” virus in solution. Since the typical size of the viruses is 20-100 nm a peak is expected in the imaginary part of the ac-susceptibility for frequencies within the 100-1000 Hz range, which is easily accessible with standard ac-susceptibility measurements. Following this initial characterization, the ac-susceptibility of the magnetic viruses is determined after they are bound to target molecules. Binding the target molecules to the virus increases the hydrodynamic radius of the virus and results in a decrease of the frequency for the peak in the imaginary part of the ac-susceptibility. This will demonstrate that biosensing is feasible through the measurement of dynamic properties of magnetic viruses.

Monolayers of magnetic viruses are needed to integrate the magnetic virus into a “laboratory on a chip” concept. The anchor of a ligand-coated ferromagnetic nanoparticle (phage displayed magnetic virus) via a linker molecule on the surface comprises a simple oscillating system. This oscillator can be driven by an external magnetic field gradient and its resonance frequency should be determined by the elastic properties of the linker molecule and the magnetic virus mass. If the molecules cover a large enough area, then this resonance frequency can be characterized through standard ac-susceptibility measurements with varying frequencies. While the magnetic moment of an individual 10 nm magnetite particle is too small (m=2.5×10⁻¹⁶ emu) to be detected directly, it is quite possible to detect even a single monolayer covering a substrate (i.e. m=10⁻⁴ emu for a 10×5 mm² substrate, while the sensitivity of a typical ac-susceptibility is 10⁻⁷-10⁻⁸ emu). This allows determining the characteristic resonant frequencies for different bio-molecule and magnetic nanoparticle combinations.

For the ac susceptibility measurements there are two approaches. The first is to measure the ac susceptibility with the remanent moment of the viruses perpendicular to the ac field. This will result in an oscillating torque, which can be used to probe the mechanical resonant frequencies. The other approach is similar to alternating gradient magnetometry [Flanders, 1988], which already has been demonstrated to reach a sensitivity of 10⁻¹² emu [M. Todorovic et al. 1998] and has theoretically the potential of even higher sensitivity [G. A. Gibson et al., 1991]. In this case an oscillating magnetic field gradient is used to excite the mechanical resonance.

Based on the assumption that the SAM of the phage displayed magnetic virus will act as an oscillator, novel sensor platforms are developed for the detection of target bio-molecules specific to the virus, since any molecule binding to the magnetic nanoparticle should modify the resonant oscillating frequency in a characteristic way. Accordingly, the above 2D assemblies of “magnetic viruses” are adapted to sensor devices as schematically depicted.

Since the magnetic viruses are part of the SAM, the intrinsic signal of resonant frequency shift comes from the increase of magnetic virus's mass due to the attachment of target molecules. There is no need for tagging the target molecules—the binding sites on the magnetic viruses have been selected for both high affinity and high specificity. The sensing mechanism has an internal check for integrity: A malfunction of the sensor will be recognized by an absence of any resonance signal.

Single Molecule Detection

The approach of biomolecule sensing through ac magnetic properties can in principle be extended to single-molecule detection by using a force detection approach. Magnetic resonance force microscopy [J. A. Sidles et al., 1995], where the magnetic resonance signal is detected by a force cantilever instead of the conventional inductive detection. This allows for exceptionally high sensitivity, which in theory is sufficient to detect magnetic resonance from even a single electron. In fact, cantilever magnetometry has already demonstrated a sensitivity down to 10⁻¹⁶ emu, [B. C. Stipe et al., 2001] which is equivalent or even below the magnetic moment that expected for a single magnetic virus.

By attaching a single magnetic virus with an elastic linker molecule to the force detection cantilever, there are two pathways for detecting a change in the mechanical resonance frequency upon binding of the target molecule. The first is to superimpose a fixed magnetic field gradient onto the oscillating gradient. This should result, during the mechanical oscillation of the magnetic virus, in a net force on the cantilever, which would be maximized for the resonance frequency of the magnetic virus/elastic linker combination. The second possibility is to use an oscillating external magnetic field for parametric mode coupling between the mechanical vibration modes of the cantilever force detector and the magnetic virus/elastic linker combination. [W. M. Dougherty et al., 1996]

Alternatively to the force detection concept, there are concepts where the mechanical motion of the magnetic viruses is inductively coupled to micropatterned pick-up loops. By using two small microfabricated and compensating detection loops, a voltage due to the motion of the magnetic virus can be detected, if only one of the loops contains a tethered virus. The induced voltage will depend on many parameters, which are difficult to estimate (such as the resonance frequency of the sensor). However, with favorable assumptions (i.e., 40 nm magnetite core of magnetic virus, 100 nm diameter of microfabricated loop, 1% displacement of virus at 1 kHz) an induced voltage signal of 1 pV-1 nV can be expected. The advantage of this integrated induction coil detection approach would be that it potentially could give rise to extremely compact sensors.

Phage-Display

In phage-display, ligands (or receptors) such as antibody fragments, cDNA encoded segments, or combinatorial peptides chains are expressed as fusions to a capsid protein present on the surface of viral particles. Libraries of millions to billions of phage particles, each displaying a different fusion protein, can then be screened by affinity selection for those members displaying the desired binding. Phage display works well because: (1) the peptide or proteins which are expressed on the surface of the viral particles are accessible for interactions with their targets; (2) the recombinant viral particles are stable; (3) the viruses can be amplified, and (4) each viral particle contains the DNA encoding the recombinant genome, thereby providing a physical linkage between the genotype and phenotype. Phage libraries are conveniently screened by isolating viral particles that bind to targets, plaque-purifying the recovered phage, and sequencing the phage DNA inserts. Usually three rounds of affinity selection are sufficient to isolate the binding phage; such a phage is confirmed by an enzyme linked immunoabsorbant assay (ELISA).

When phage-display combinatorial peptide libraries are screened by affinity selection with a particular target protein, in many cases it is possible to identify, from the affinity selected peptides, members with a sequence that closely resembles segments (epitopes) of a natural interacting partner of the protein. A practical consequence of this phenomenon, termed “convergent evolution” is that one can search whole genome databases for proteins containing segments that match consensus sequences shared by the selected peptides, and then experimentally determine whether or not they interact with the target. Combinatorial peptide libraries have proven useful in defining the optimal ligand preferences of protein interaction modules, such as EH, PDZ, SH2, SH3, and WW domains, the heterodimeric G protein β and γ subunit, the catalytic subunit of protein phosphatase 1 (PP1c), the estrogen receptor and the ubiquitin ligase, DM2. Thus, screening a phage-displayed combinatorial peptide library has proved to be a fruitful means of isolating a peptide ligand to a protein target.

### PUBLICATIONS CITED

These publications are incorporated by reference to the extent they relate materials or methods to practice the claimed invention.

Adams, S. R. et al. “New biarsenical ligands and tetracysteine motifs for protein labeling in vitro and in vivo: synthesis and biological applications”. J. Am. Chem. Soc. 124, 6063-6076 (2002).

Allen, M., Willits, D., Mosolf, J., Young, M. & Douglas, T. Protein cage constrained synthesis of ferrimagnetic iron oxide nanoparticles. Advanced Materials (Weinheim, Germany) 14(21), 1562-1565 (2002).

Bartlett J M. “Fluorescence in situ hybridization: technical overview.” Methods Mol Med. 2004; 97:77-87.

Casjens, S., “Nucleic acid packaging by viruses”. In Virus Structure and Assembly (Casjens, S., Ed.), pp. 75-147. Jones and Bartlett, Boston. 1985

Cerritelli, Mario E. Cheng, Naiqian, Rosenberg, Alan H., McPherson, Catherine E., Booy, Frank P. and Steven, Alasdair C. “Encapsidated Conformation of Bacteriophage T7 DNA” Cell, 91, 271-280, 1997.

Chiu, W., Burnett R. M. & Garcea, R. L. Structural Biology of Viruses Oxford University Press, (1997).

Chung, S. H., Hoffmann, A., Bader, S. D., Chen, L., Liu, C., Kay, B. & Makowski, L. Biological sensors based on Brownian relaxation of magnetic nanoparticles. Appl. Phys. Lett. 85, 2971-2973 (2004).

Connolly, J. and T. G. St Pierre, J. Magnet. Magnet. Mater. 225, 156 (2001)

Debye, P. Polar Molecules, Chemical Catalog Company, New York, (1929).

Diamandis, Eleftherios P. “Europium and terium Chelators as candidates substrates for enzyme-labeled Time-resolved fluorimetric Immunoassays” Analyst 117, 1879-1884 (1992)

Dougherty, W. M., K. J. Bruland, J. L. Garbini, and J. A. Sidles, Meas. Sci. Technol. 7, 1733 (1996)]

Douglas, T. & Young, M. Host-guest encapsulation of materials by assembled virus protein cages. Nature (London) 393(6681), 152-155 (1998).

Douglas, T., Allen, M. & Young, M. Self-assembling protein cage systems and applications in nanotechnology. Biopolymers 8, 405-426 (2003).

Flanders, P. J., J. Appl. Phys. 63, 3940 (1988)

Gibson, G. A. and S. Schultz, J. Appl. Phys. 69, 5880 (1991)]

Huter, V., Szostak, M. P., Gampfer, J., Prethaler S., & Lubitz, W. Bacterial ghosts as drug carrier and targeting vehicles. Journal of controlled release 61, 51-63 (1999).

Ivanovska, I. L., de Pablo, P. J., Ibarra, B., Sgalari, G., MacKintosh, F. C., Carrascosa, J. L., Schmidt, C. F. & Wuite, G. J. Bacteriophage capsids: Tough nanoshells with complex elastic properties Proc. Natl. Acad. Sci. U S A. 101(20), 7600-5 (2004).

Kasanov, J.; Pirozzi, G.; Uveges, A. J., and Kay, B. K. “Defining the ligand specificity of WW domains using phage-displayed combinatorial peptides.” Chem. Biol. 2001, 8, 231-241.

Kay, B. K.; Kasanov, J.; and Yamabhai, M. “Screening phage-displayed combinatorial peptide libraries.” Methods 2001, 24, 240-246.

Kobayashi, Y., Horie, M., Konno, M., Rodriguez-Gonzalez, B. & Liz-Marzan, L. M. Preparation and properties of silica-coated cobalt nanoparticles. Phys. Chem. B. 107(30), 7420-7425 (2003).

Liu, Chinmei, Sutton, April, Yang, Funing, Chung, Seok-Hwan, Jin, Qiaoling, Hoffmann, Axel, Kay, Brain, Bader, Samuel D., Makowski, Lee, Chen, Liaohai “Magnetic and Bio-Functional Phage”, in preparation.

Liu, C., Jin, Q., Sutton, A. & Chen, L. A novel fluorescent probe: Europium complex hybridized T7 phage. paper submitted.

Mann, S. Biomimic approaches in material science. VCH, New York (1996).

Mao, C., Solis, D. J., Reiss, B. D., Kottmann, S. T., Sweeney, R. Y., Hayhurst, A., Georgiou, G., Iverson, B. & Belcher, A. M. Virus-based toolkit for the directed synthesis of magnetic and semiconducting nanowires. Science 303(5655), 213-7 (2004).

Nagai, T. et al, “A variant of yellow fluorescence protein with fast and efficient maturation for cell-biological applications” Nature Biotechnol. 20, 87-90 (2002).

Ormö, Mats Cubitt, Andrew B., Kallio, Karen, Gross, Larry A., Tsien, Roger Y. and Remington, S. James “Crystal Structure of the Aequorea Victoria Green Fluorescent Protein” Science 6 September 1996; 273: 1392-1395.

Pankhurst, Q. A., Connolly, Jones, J., S. K., & Dobson, J. Applications of magnetic nanoparticles in biomedicine. J. Phys. D: Appl., 36, R167-R181 (2003).

Raines, R. T.; Mccormick, M.; VanOosbree, T. R.; and Mierendorf, R. C. Methods of Enzymology, 2000, 326, 362-376.

Raines, R. T., Mccormick, M., VanOosbree, T. R. & Mierendorf, R. C. The s-tag fusion system for protein purification. Methods of Enzymology 326, 362-376 (2000).

Resnick, D., Gilmore, K., Idzerda, Y. U., Klem, M., Smith, E. & Douglas, T. Modeling of the magnetic behavior of gamma-Fe2O3 nanoparticles mineralized in ferritin. Journal Of Applied Physics 95(11), pt.2, 7127-7129 (2004).

Rodi, D. J., and Makowski, L. “Phage-display technology—finding a needle in a vast molecular haystack.” Current Opinion in Biotechnology 1999, 10(1), 87-93.

Roeder, Shirleen and Sadowski, Paul “Bacteriphage T7 Morphogenesis: Phage-Related particles in Cells infected with wild-Type and Mutant T7 phage” Virology 76, 263-285 (1977).

Sarikaya, M., Tamerler, C., Jen, A. K., Schulten, K. & Baneyx, F. Molecular biomimetics: nanotechnology through biology. Nat. Mater. 2(9), 577-85 (2003)

Selvin, Paul R. “Principles and biophysical applications of lanthanide-based probes”. Annu Rev Biophys Biomol Struct. 2002; 31:275-302.

Shliomis, M. I. Sov. Phys.-Usp. 17, 153-169 (1974).

Sidles, J. A., J. L. Garbini, K. J. Bruland, D. Rugar, O. Züger, S. Hoen, and C. S. Yannoni, Rev. Mod. Phys. 67, 249 (1995)

Stipe, B. C., H. J. Mamin, T. D. Stowe, T. W. Kenny, and D. Rugar, Phys. Rev. Lett. 86, 2874 (2001)

Studier, William “Bacteriophage T7” Science. 1972 Apr 28;176(33):367-76.

Studier, F. W. Bacteriophage T7 Science. 176(33):367-76 (1972).

Studier, William “The genetics and physiology of bacteriophage T7” Virology. 1969 November; 39(3):562-74.

Todorovic, M. and S. Schultz, Appl. Phys. Lett. 73, 3595 (1998)]

Zhang, S. Fabrication of novel biomaterials through molecular self-assembly. Nature Biotechnology 21(10), 1171-8 (2003).

Zhang, Jin, Campbell, Robert E., Ting, Alice Y., Tsien, Roger Y. “Creating New Fluorescent Probes for Cell Biology” Nature Reviews Molecular Cell Biology 3, 906-918 (01 Dec. 2002)

We claim:

1. A nanosized biological container comprising:
(a) a viral capsid protein shell independent of an endogenous core; and
(b) an exogenous functional core.

2. The nanosized biologial container of claim 1 wherein the viral capsid protein shell is a T7 bacteriophage ghost.

3. The nanosized biological container of claim 1, wherein the exogenous functional core is selected from the group consisting of a fluorescent, magnetic, x-ray absorbent, exogenous nucleotide and radioactive particle.

4. The nanosized biological container of claim 3, wherein the magnetic particle is cobalt.

5. The nanosized biological container of claim 3, wherein the fluorescent particle is a lonthonide complex.

6. The nanosized biological container of claim 3, wherein the capsid protein shell comprises ligands that are covalently bound to the external surface of the capsid shell.

7. The nanosized biological container of claim 6, wherein the capsid protein shell comprises a fusion protein, said fusion protein comprising a ligand.

8. The nanosized biological container of claim 7, wherein the ligand is an antibody.

9. The nanosized biological container of claim 6, wherein the capsid protein shell comprises a fusion protein, said fusion protein comprising a ligand useful for affinity purification of said container.

10. The nanosized biological container of claim 3, wherein the capsid protein shell is bound to a solid support via a linker.

11. A biosensor comprising the nanosized biological container of claim 3, wherein said container further comprises a ligand, covalently bound to the external surface of the capsid shell.

12. The biosensor of claim 11 wherein the nanosized biological container comprises a magnetic exogenous functional core, and said nanosized biological container is covalently bound to a solid support via a linker.

13. A method of preparing a nanosized biological container, the method comprising:
(a) contacting phage with a sodium sulfate solution in the presence of DNAase; and
(b) purifying phage ghosts by centrifugation using cesium chloride density gradients.

14. A method of preparing a nanosized biological container, the method comprising:
(a) contacting phage with with alkaline buffer;
(b) isolating the capsid proteins;
(c) renaturing the capsid proteins to form phage ghosts.

15. A method of placing an exogenous core into a viral capsid, the method comprising:
(a) obtaining a solution of phage ghosts; and
(b) mixing a solution of core particles with the solution of phage ghosts.

16. A method of adding ligands to the surface of a natural viral capsid protein shell from which endogenous DNA or RNA has been removed, the method comprising:
(a) obtaining a solution of phage ghosts displaying ligands; and
(b) selecting the phage ghosts with ligands.

17. A method of manufacturing uniform nanosized particles with uniform size distribution, the method comprising:
(a) obtaining a solution of phage ghosts displaying ligands; and
(b) selecting the phage ghosts with ligands.

18. A method of performing an enzyme linked immunosorbant assay (ELISA), the method comprising:
(a) preparing microtiter plates containing protein;
(b) adding nanosized containers of the present disclosure to the wells;
(c) adding labelled protein to the wells; and
(d) interpreting the results to determine binding.
